(19) United States
(12) Patent Application Publication
(10) Pub. No.: US 2011/0257436 A1
(43) Pub. Date: Oct. 20, 2011

(54) PROCESS FOR THE MANUFACTURE OF EASILY DISPERSIBLE, SOLID N'-HYDROXY-N-CYCLOHEXYL-DIAZENIUM OXIDE SALTS

(75) Inventors: Darren Michael Hodgkinson, Nottingham (GB); James Bullock, Grantham (GB); Mohammed Shoaib Qureshi, Nottinghamshire (GB)

(73) Assignee: BASF SE, Ludwigshafen (DE)

(21) Appl. No.: 13/139,622

(22) PCT Filed: Dec. 10, 2009
(86) PCT No.: PCT/EP2009/066801

§ 371 Date: Jun. 14, 2011

(30) Foreign Application Priority Data
Dec. 15, 2008 (EP) 08171664.9

(51) Int. Cl. C07C 291/04

(52) U.S. Cl. 564/298; 564/297

(57) ABSTRACT

Process for the manufacture of solid Mⁿ⁺(HDO)ₙ salts wherein HDO is the anion of N′-hydroxy-N-cyclohexyldiazenium oxide, and Mⁿ⁺ is a metal cation by precipitation with an acid from an alkaline solution of an HDO-salt with monovalent cations in the presence of amines and drying under mild conditions.

This invention relates to a process for the manufacture of solid Mⁿ⁺(HDO)ₙ wherein HDO is the anion of N′-hydroxy-N-cyclohexyldiazenium oxide, and Mⁿ⁺ is a metal cation by precipitation with an acid from an alkaline solution of an HDO-salt with monovalent cations in the presence of amines and drying under mild conditions.

For many years, it has been known (e.g. disclosed by DE-A 10 24 743) that metal salts, such as Ca-, Ba-, Al-, Pb-, Ag-, Cu-, Fe-, Ni-, or Zn-salts of N-alkyl-N-nitrohydroxyl-amines (also referred to as N′-hydroxy-N-alkyl diazenium oxides) are effective for inhibiting fungal growth.

EP-A 358 072 discloses a method of controlling organisms which grow under moist conditions, such as algae and lichen, by treatment with certain metal salts, notably copper, aluminum or tin salts, or amine salts of N′-hydroxy-N-cyclohexyldiazenium oxide. The biocidal active component may be incorporated directly into a polymer matrix, such as a polymer foil, or may be added to aqueous or organic solvent based media to be protected, such as paints, especially antifouling paints.

The Cu-salt of N′-hydroxy-N-cyclohexyldiazenium oxide Cu(HDO)₂ has only a poor solubility in water of only around 30 ppm. However, it is often necessary to use Cu(HDO)₂ in aqueous media.

WO 2005/044010 discloses the use of the Cu-salt of N′-hydroxy-N-cyclohexyl-diazenium oxide Cu(HDO)₂ for combating and/or killing bacteria, mould, yeast, and algae in industrial materials such as lacquers, wood, coating materials or anti-fouling coatings using a formulation of Cu(HDO)₂ and a diluent. The formulation may additionally contain surfactants for enhancing the solubility of Cu(HDO)₂ in water.

It is also known in the art to add complex forming compounds to Cu(HDO)₂ formulations in order to enhance the solubility of Cu(HDO)₂. U.S. Pat. No. 4,143,153 discloses a mixture of Cu(HDO)₂ and water soluble complex forming compounds for the protection of wood. U.S. Pat. No. 4,761,179 and U.S. Pat. No. 5,187,194 disclose similar formulations which comprise a polyamine and a complex forming carboxylic acid or polymeric polyamines as complex forming compounds.

Such formulations comprising Cu(HDO)₂ solubilized by using certain additives may be used for the protection of materials, such as paints, coatings and coated substrates. However, the additives used can also help to leach Cu(HDO)₂ out from the coating and/or substrate which is highly undesirable. Furthermore, soluble Cu(HDO)₂ has an intense blue color which may also be undesirable in certain applications, in particular for the use of Cu(HDO)₂ in paints, coatings or coated substrates for decorative purposes.

It is known in the art from DE 1 024 743 to grind solid Cu(HDO)₂ to a fine powder and to apply the powder as a dispersion in water. However, such a grinded powder is difficult to disperse homogeneously in liquid formulations, in particular aqueous formulations, such as for instance paints or coatings.

It was an object of the invention to provide an improved process for the manufacture of solid salts of N′-hydroxy-N-cyclohexyldiazenium oxide which are easily to disperse in liquid formulations, in particular in aqueous formulations.

According to a first aspect, the invention relates to a process for the manufacture of solid Mⁿ⁺(HDO)ₙ salts, wherein HDO is the anion of N′-hydroxy-N-cyclohexyldiazenium oxide, Mⁿ⁺ is a cation with the exception of alkali metal cations, and n has a value of 1 to 4 comprising at least the following steps:
(1) Providing an aqueous mixture comprising at least
   an aqueous soluble Mⁿ⁺-salt,
   a compound M(I)(HDO), wherein M(I) is at least one monovalent cation in an amount of n to 1.5 n moles per mole of Mⁿ⁺, and wherein the cation is selected from the group of H⁺, alkali metal ions, ammonium ions N(R¹)₄⁺ and/or phosphonium ions P(R²)₄⁺, wherein R¹ and R² are selected—independently of each other—from H and hydrocarbon substituents comprising 1 to 30 carbon atoms and/or hydroxy-substituted hydrocarbon substituents comprising 1 to 30 carbon atoms, with the proviso that the M(I) cations and the Mⁿ⁺-cations used are different,
   at least one aqueous soluble amine in an amount of 1 to 6 moles of amino groups per mole of Mⁿ⁺,
and adjusting the pH-value of the aqueous mixture to 10 to 14,
(2) mixing the components at a temperature from 30 to 70° C. to obtain a substantially homogeneous mixture,
(3) precipitating solid Mⁿ⁺(HDO)ₙ salt by cooling to a temperature below a temperature of 30° C. and adjusting the pH-value of the mixture using an acid to a pH-value from 5 to 9.5,
(4) Collecting the formed precipitate of Mⁿ⁺(HDO)ₙ,
(5) Drying the precipitate at a temperature below 45° C.

According to a second aspect, the invention relates to solid Mⁿ⁺(HDO)ₙ salts, wherein HDO is the anion of N′-hydroxy-N-cyclohexyldiazenium oxide, Mⁿ⁺ is a metal cation with the exception of alkali metal cations and n has a value of 1-4 having a particle size D50 from 30 to 75 μm.

According to a third aspect, the invention relates to the use of such solid Mⁿ⁺(HDO)ₙ salts for the protection of paints, coatings, construction materials, plastics and paper, leather, textiles, polymeric materials or surfaces.

Details of the invention now follow.

The process according to the invention yields solid Mⁿ⁺(HDO)ₙ salts wherein HDO is the anion of N′-hydroxy-N-cyclohexyldiazenium oxide having the following formula (I)

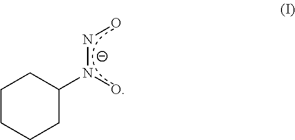

The number n has a value of 1 to 4, preferably 2 or 3, and most preferred n is 2. Mⁿ⁺ is a cation with the exception of alkali metal cations. The cation may be a metal cation but also other cations such as ammonium cations or phosphonium cations may be used.

Preferred metal cations include alkaline earth metal cations such as Mg²⁺, Ca²⁺ or Ba²⁺, Al³⁺, and transition metals, in particular first row transition metals such as Ti³⁺, Ti⁴⁺, Fe²⁺, Fe³⁺, Co²⁺, Ni²⁺, Cu²⁺, and Zn²⁺.

In a preferred embodiment of the invention n is 2 or 3 and Mⁿ⁺ is selected from the group of Cu²⁺, Zn²⁺, Ni²⁺, Co²⁺, Ca²⁺, and Al³⁺, more preferably Ca²⁺, Zn²⁺, and Cu²⁺, and most preferred Mⁿ⁺ is Cu²⁺.

In step (1) of the process according to the invention an aqueous mixture comprising at least an M(I)(HDO) compound, an amine and an aqueous soluble Mⁿ⁺-salt is provided.

As Mⁿ⁺-salts any kind of soluble salts of the Mⁿ⁺-cations may be used. As suitable salts there may be mentioned in particular carbonate, sulphate, nitrate, phosphate, halides, oxides or silicates. Preferred salts are sulphates. It is of course possible to use a mixture a two or more different Mⁿ⁺-salts. If the salts do not readily dissolve in water to obtain an aqueous solution, optionally an acid, such as sulphuric acid may be used to dissolve the salt in water.

In the M(I)(HDO) compounds M(I) is at least one monovalent cation selected from the group of H⁺, alkali metal ions, ammonium ions, and phosphonium ions with the proviso, that the M(I) cations and the Mⁿ⁺-cations used in the process are different.

Suitable ammonium ions are of the formula N(R¹)₄⁺, wherein the R¹ are selected—independently of each other—from H and hydrocarbon substituents comprising 1 to 30 carbon atoms, preferably 1 to 10 carbon atoms which may comprise additional functional groups such as OH-groups. Preferably, R¹ is selected from the group of H, an unsubstituted, linear alkyl group with 1 to 4 C-atoms or a linear monohydroxy alkyl group with 2 to 4 C-atoms.

Suitable phosphonium ions are of the formula P(R²)₄⁺, wherein R² is selected—independently of each other—from H and hydrocarbon substituents comprising 1 to 30 carbon atoms, preferably 1 to 10 carbon atoms and which may comprise additional functional groups such as OH-groups. Preferably, at least one, preferably at least 2 of the substituents R² are not hydrogen. Preferably, R² is an aliphatic or aromatic hydrocarbon group with 1 to 10 carbon atoms. As an example the tetraphenylphosphonium ion may be mentioned as a suitable ion.

Preferably, M(I) is selected from the group of Na⁺, K⁺, and NH₄⁺, and most preferred M(I) is K⁺. An aqueous solution of K(HDO) is commercially available, e.g. under the trademark Protectol® KD.

It is advisable to use the M(I)(HDO) compounds in a slight excess in relation to the Mⁿ⁺-salts. In general, the amount of M(I)(HDO) compounds is from n to 1.5 n moles per mole of Mⁿ⁺, preferably n to 1.25 n, and most preferred from 1.05 n to 1.2 n; i.e. for n=2 the numbers are 2 to 3 moles per mole of M²⁺, preferably 2 to 2.5 moles, and most preferred from 2.1 to 2.4.

For the present invention any kind of aqueous soluble amine may be used. Of course, a mixture of two or more different amines may be used. Suitable amines may comprise primary, secondary or tertiary amino groups. Preferably, the amines comprise only primary and/or secondary amino groups. Preferably, the amine is an aliphatic diamine or a an aliphatic triamine. Examples of suitable amines include methylamine, dimethylamine, ethanolamine, cylic amines such as pyrrolidone, tertiary amines such as trimethylamine or aromatic amines such as aniline, ethylene diamine, 1,2-propylene diamine, 1,3-diamino propane, or diethylene triamine. Examples of preferred amines include ethylene diamine, 1,2-propylene diamine, 1,3-diamino propane, or diethylene triamine. Most preferably, ethylenediamine may by used as the amine.

The amount of amines is from 1 to 6 moles of amino groups per mole of Mⁿ⁺, preferably from 1.5 to 4 moles and most preferred from 2 to 3 moles.

The sequence of adding the components to each may be chosen by the skilled artisan. Advantageously, one may provide as a first step a mixture of a portion of the M(I)(HDO) compound in water (e.g. 30 to 50% of the total amount) and add the amine, then add the Mⁿ⁺-salt and thereafter add the remaining portion of the M(I)(HDO) compound, however the invention is not limited to said order.

The pH-value of the mixture is from 10 to 14, preferably from 11 to 13. If necessary the pH-value may be adjusted to said numbers using suitable bases such as NaOH or KOH. Whether an adjustment is necessary depends on the nature of the starting material(s). If M(I) in the M(I)(HDO) compounds is H⁴ or if an acidic solution of a Mⁿ⁺-salts is used then an adjustment will usually be necessary, while a solution of KHDO is strongly alkaline so that an adjustment may not be necessary.

The total amount of all components of the mixture except water is from 5 to 40% by weight based on the total amount of all components of the aqueous mixture, preferably from 20 to 40% by weight, and most preferred from 25 to 35% by weight.

In step (2) of the process according to the invention, the mixture of step (1) is mixed in order to obtain a substantially homogeneous mixture. “Substantially homogeneous” shall mean that most of the components used in step (1) are preferably fully dissolved in the water, however is also sufficient if the components form a homogeneous suspension. Mixing may be done by known techniques such a stirring or shaking the mixture, preferably by stirring.

The mixing step (2) is performed at a temperature above room temperature. Mixing may be done at a temperature from 30 to 70° C. A preferred temperature range for mixing is from 30° C. to 55° C., more preferred 35 to 55° C. and most preferred from 45 to 50° C. Mixing may be performed overnight, however in general mixing the solution shall be performed for 1 to 6 hours, preferably 2 to 6 hours and most preferred 4 to 5 hours.

After obtaining a substantially homogenous solution in step (2), in step (3) of the process solid Mⁿ⁺(HDO)ₙ salts are precipitated from the solution by cooling to a temperature below 30° C. and by adjusting the pH-value of the solution to 5 to 9.5 using an acid. Preferably, the pH-value is adjusted to 6.5 to 8.5 and most preferred it is adjusted to 6.5 to 7.5. The mixture may be cooled to a temperature from 0° C. to 25° C., preferably from 15 to 25° C. It is possible to cool the solution first and then to adjust the pH-value, to perform the steps in reverse order or to do the steps simultaneously.

Examples of suitable acids include organic or inorganic acids such as H₃PO₄, acetic acid, formic acid, propionic acid, 2-ethylhexanoic acid, citric acid, oxalic acid, sulphonic acid, sulphamic acid, lactic acid, gluconic acid, sulphuric acid, nitric acid or halide based acids such as HCl or HBr. A preferred acid is H₃PO₄.

After precipitation the solid Mⁿ⁺(HDO)ₙ salts are collected by usual techniques for the separation of solids form liquids, e.g. by filtration or centrifugation. Furthermore, the obtained solids may be washed, e.g. to remove excess amounts of M(I)(HDO) compounds and/or the amine. In technical scale, collection and washing the precipitate may be performed for instance by using a filter press.

Finally the precipitate of solid Mⁿ⁺(HDO), salts is dried at temperatures below 45° C., preferably below 35° C., more preferred below 25° C. and most preferred below 20° C. Drying the Mⁿ⁺(HDO)ₙ salts at higher temperatures may result in products with deteriorated dispersing characteristics.

Any drying method may be used which does not involve temperatures above the cited limits, e.g. drying the Mⁿ⁺(HDO)ₙ salts in the presence of suitable desiccants and/or techniques employing reduced pressure. In a preferred embodiment of the invention drying may be done by freeze drying.

The process according to the invention yields in a homogeneous fine powder of solid Mⁿ⁺(HDO)ₙ which does not contain coarse particles. The powder obtained is easily dispersible in aqueous and non-aqueous media.

Therefore, in a further embodiment of the present application relates to solid Mⁿ⁺(HDO)ₙ particles which are available by said process having a particle size D50 from 30 to 75 μm, preferably from 35 μm to 70 μm and most preferred from 40 μm to 60 μm.

Preferably, the particle size D10 is from 5 to 25 μm, more preferred from 10 to 20 μm, and preferably, the particle size D90 is from 80 to 200 μm, more preferred from 90 to 150 μm.

The D10, D50 and D90 represent the median or the 10^{th}, 50^{th}, and the 90^{th} percentile of the particle size distribution (volume distribution), respectively. That is, the D50 (D10/D90) is a value on the distribution such that 50% (10%/90%) of the particles have a particle size of this value or less. The volume averaged particle size distribution may be measured with an aqueous dispersion of the Mⁿ⁺(HDO)ₙ by techniques known to the skilled artisan, e.g. by laser diffraction techniques.

In a preferred embodiment, the Mⁿ⁺(HDO)ₙ particles are selected from the group of Cu(HDO)₂, Zn(HDO)₂, Ni(HDO)₂, Co(HDO)₂, Ca(HDO)₂ and Al(HDO)₃, and most preferred the solid salt is Cu(HDO)₂.

The particle size distribution is excellent for dispersing the particles in media to be protected. The fraction of coarse particles which are only difficult to disperse is very low but also the amount of fine particles with a particle size of only a few μm or even less which are difficult to disperse either is low.

In a third embodiment of the present invention the solid Mⁿ⁺(HDO)ₙ particles according to the invention may be used as a microbicidal active component basically in the same applications as conventionally prepared Mⁿ⁺(HDO)ₙ, in particular for combating the growth of microorganisms such as bacteria, fungi, and algae. In particular it may be used for combating the microorganisms cited in WO 2005/044010, page 4, lines 25 to 34.

It may be used for the protection of industrial materials, such as those cited in WO 2005/044010, page 7, lines 15 to 25, and in industrial processes such as those cited in WO 2005/044010, page 7, lines 27 to 30.

In particular, the solid Mⁿ⁺(HDO)ₙ salts prepared according to the present invention may be used for the protection of paints, lacquers, coatings, construction materials, plastics, paper, leather, textiles, polymeric materials or surfaces, most preferred for the protection of paints, lacquers, and coatings. The particles may be easily dispersed in paint and/or lacquer formulations which are thereafter applied to surfaces.

In a preferred embodiment, the solid Mⁿ⁺(HDO)ₙ used as microbicides are selected from the group of Cu(HDO)₂, Zn(HDO)₂, Ni(HDO)₂, Co(HDO)₂, Ca(HDO)₂ and AI(HDO)₃, and most preferred the solid salt is Cu(HDO)₂.

Embodiments of the invention will now be described in more detail with reference to the following Examples.

### Preparation of Solid Cu(HDO)₂

As starting materials the following components were used:

| | | |
|---|---|---|
| K(HDO) (solution in water, contents 30% by weight) | 690 g | 1.2 mole |
| ethylene diamine | 75 g | 1.2 mole |
| CuSO₄•5H₂O | 130 g | 0.5 mole |
| De-mineralized water | 222 g | |

Ethylene diamine, de-mineralized water and 307 g of the K(HDO) solution were placed in a beaker with stirring, the Copper sulphate added and the mixture stirred at ambient temperature for 30 min. Thereafter, another 383 g of the K(HDO) solution were added and again stirred at ambient temperatures for 30 min. The mixture obtained had a pH value of 13 to 14. The mixture was warmed up to 50° C. and maintained for 5 hours at said temperature with stirring. Thereafter, the mixture was allowed to cool to room temperature and the pH-value of the mixture was adjusted with orthophosphoric acid to pH 7. Immediately a pale blue precipitate appeared which was filtered off and washed with 1 l of demineralized water. The solid was removed re-suspended in 1 l of water and then freeze dried.

### Yield: 184 g of Cu(HDO)₂

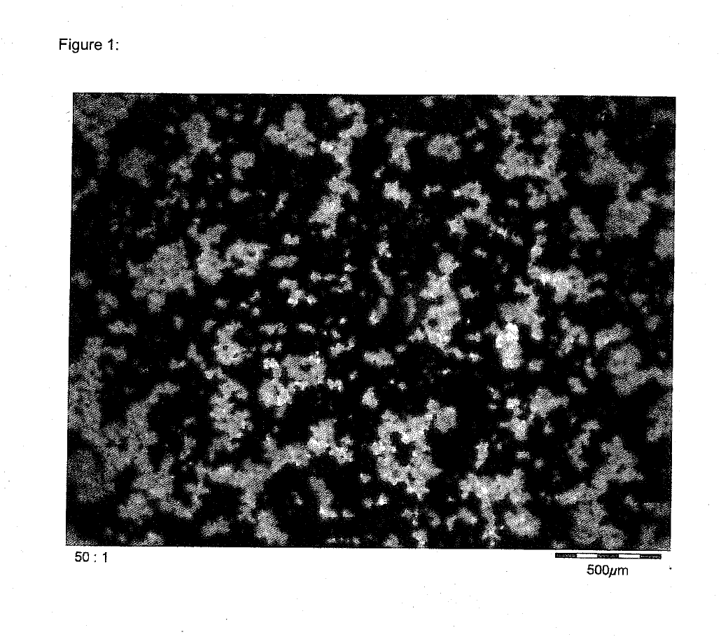

The prepared Cu(HDO)₂ sample was analyzed using an optical microscope. FIG. 1 shows a micrograph of the sample.

Furthermore, a particle size distribution analysis (volume distribution) was performed using a conventional laser diffraction apparatus (Mastersizer® 2000, Fa. Malvern Instruments). For the measurement, 1 g of Cu(HDO)₂ was dispersed in 100 ml of water sample and 1 ml of a solution (1% by weight) of a nonionic surfactant (polyethylene glycole p-(1,1,3,3-tetramethylbutyl)-phenyl ether, around 9-10 ethylene oxide groups). The dispersion time was 1 min.

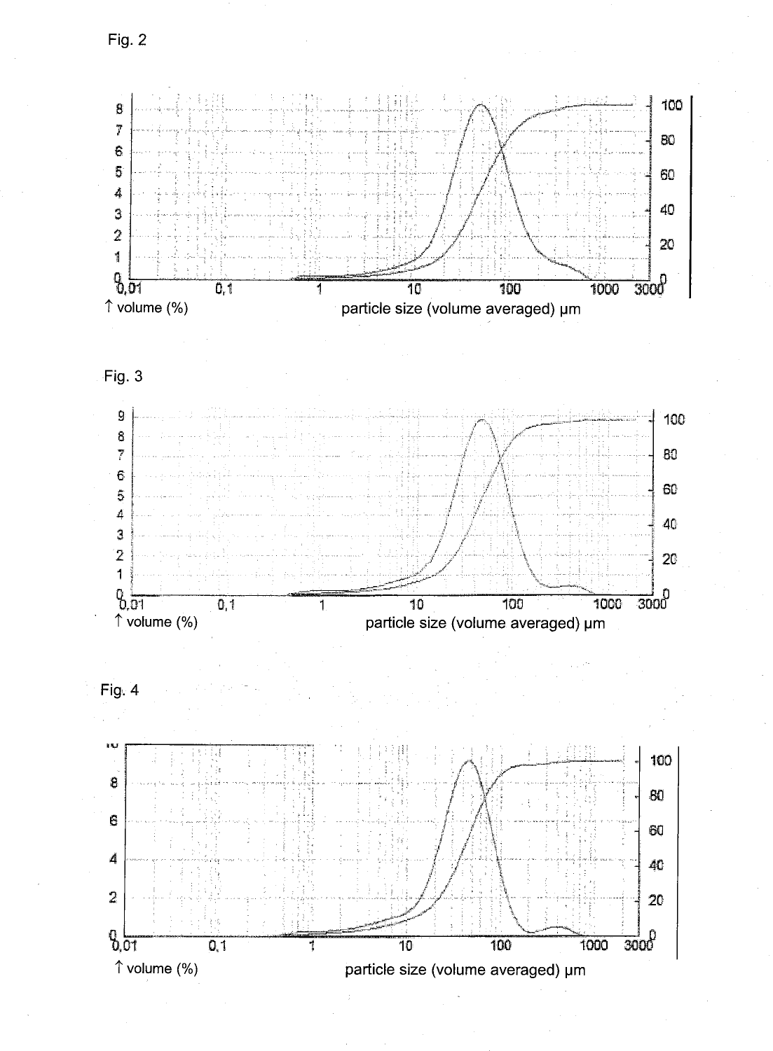

FIG. 2 shows the particle size distribution (volume distribution). The distribution is monomodal with a maximum at around 50 μm. There is only a slight shoulder at around 300 to 400 μm. The D10 value is 16 μm, D50 49 μm, and D90 is 140 μm. The particle size distribution curve is symmetrical and very close to gauss shaped curve.

FIG. 3 shows the result of the particle size distribution analysis after 10 min dispersion instead of 1 min, The intensity of the slight shoulder at 300 to 400 μm diminished a little bit. The D10 value is 14 μm, D50 is 45 μm, and D90 is 108 μm. The particle size distribution curve still is symmetrical and very close to gauss shaped curve.

FIG. 4 Fig. shows the result of the particle size distribution analysis after 30 min dispersion.

The D10 value is 12 μm, D50 is 41 μm, and D90 is 93 μm. The particle size distribution curve still is symmetrical and very close to gauss shaped curve.

### Comparative Example

A comparative sample of Cu(HDO)₂ was prepared in analogy to procedure described above, however the step of mixing for 5 h at 50° C. was omitted and drying was performed at a temperature from 60° C. to 80° C.

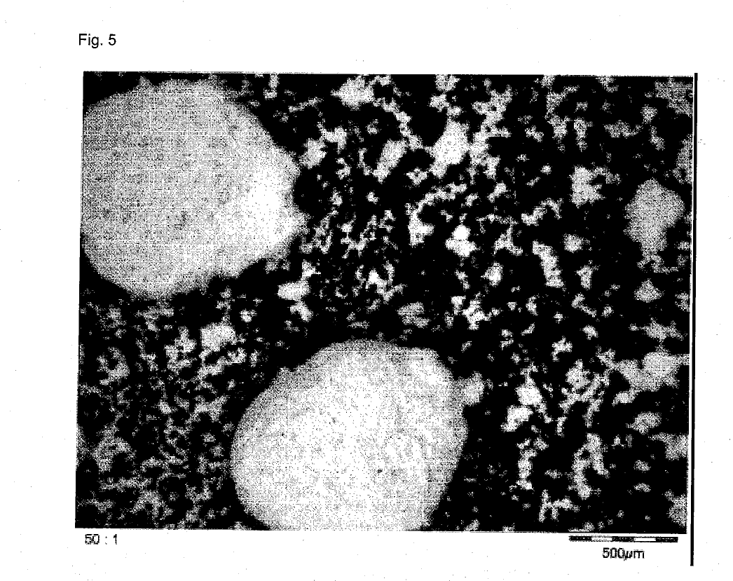

FIG. 5 shows an micrograph of Cu(HDO)₂ prepared in the comparative example. The micrograph shows a mixture of coarse and fine particles.

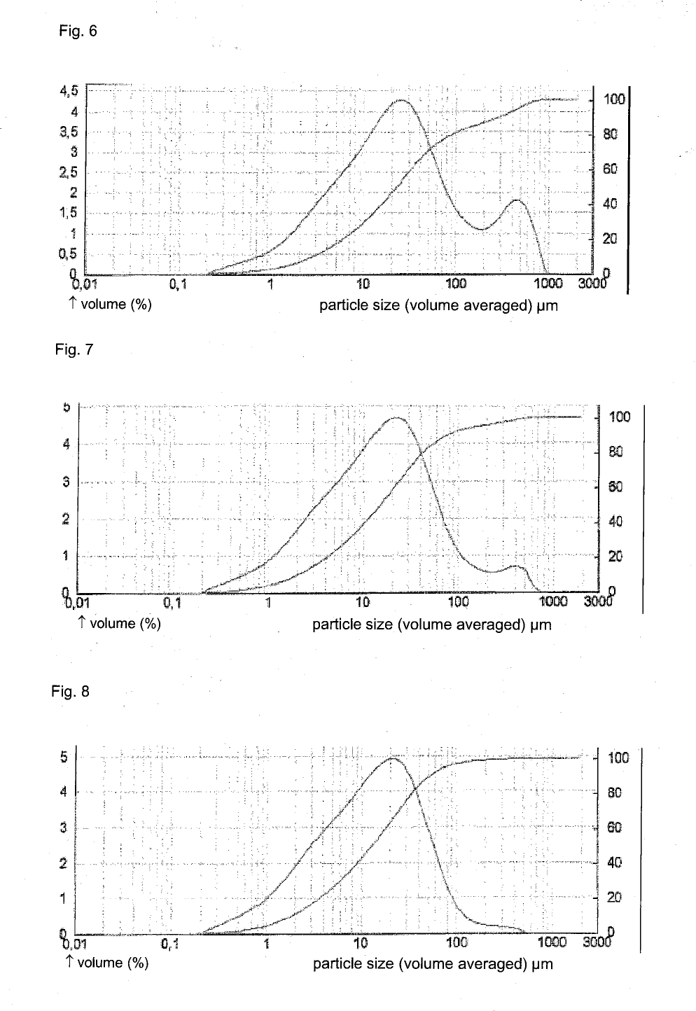

FIG. 6 shows the result of a particle size distribution analysis (volume distribution) performed under the same conditions as in the example at a dispersion time of 1 min. The distribution clearly is bimodal; i.e. also the particle distribution analysis demonstrates significant amounts of coarse particles. The maximum of the first peak is at around 25 μm and the maximum of the second peak is at around 450 μm. The D10 value is 3 μm, D50 is 23 μm, and D90 is 306 μm.

FIG. 7 shows the result of the particle size distribution analysis after 10 min dispersion instead of 1 min. The intensity of the peak at around 450 μm is less than after only 1 min dispersion. The D10 value is 2 μm, D50 15 μm, and D90 is 79 μm. However the particle size distribution is still clearly broader than in the example according to the invention and furthermore not symmetrical.

FIG. 8 shows the result of the particle size distribution analysis after 30 min dispersion. The peak at around 450 μm disappeared, however the distribution still is broad and not symmetrical. The D10 value is 2 μm, D50 12 μm, and D90 is 53 μm.

The examples and comparative examples demonstrate that the process according to the invention yields in products with a narrow, basically monomodal particle size distribution and in particles which may be easily dispersed. Longer dispersion times have only little effect.

The comparative samples according to the state of the art comprise a significant amount of coarse particles which—even after a longer dispersion time—yield only in a very broad particle size distribution. It is needless to say that such long dispersion times are at least highly undesirable if not impossible to apply for goods to be protected.

1.-16. (canceled)

17. A process for the manufacture of solid Mⁿ⁺(HDO)ₙ salts, wherein HDO is the anion of N′-hydroxy-N-cyclohexyldiazenium oxide, Mⁿ⁺ is a cation with the exception of alkali metal cations, and n has a value of 1 to 4 comprising at least the following steps:
(1) Providing an aqueous mixture comprising at least
an aqueous soluble Mⁿ⁺-salt,
a compound M(I)(HDO), wherein M(I) is at least one monovalent cation in an amount of n to 1.5 n moles per mole of Mⁿ⁺, and wherein the cation is selected from the group of H⁺, alkali metal ions, ammonium ions N(R¹)₄⁺ and/or phosphonium ions P(R²)₄⁺, wherein R¹ and R² are selected—independently of each other—from H and hydrocarbon substituents comprising 1 to 30 carbon atoms and/or hydroxy-substituted hydrocarbon substituents comprising 1 to 30 carbon atoms, with the proviso that the M(I) cations and the Mⁿ⁺-cations used are different,
at least one aqueous soluble amine in an amount of 1 to 6 moles of amino groups per mole of Mⁿ⁺,
and adjusting the pH-value of the aqueous mixture to 10 to 14,
(2) mixing the components at a temperature from 30 to 70° C. to obtain a substantially homogeneous mixture,
(3) precipitating solid Mⁿ⁺(HDO)ₙ salt by cooling to a temperature below a temperature of 30° C. and adjusting the pH-value of the mixture using an acid to a pH-value from 5 to 9.5,
(4) Collecting the formed precipitate of Mⁿ⁺(HDO)ₙ,
(5) Drying the precipitate at a temperature below 45° C.

18. The process according to claim 17, wherein the pH-value in course of step (2) is from 11 to 13.

19. The process according to claim 17, wherein in course of step (2) the solution is stirred for 1 to 6 hours at a temperature from 20 to 50° C.

20. The process according to claim 17, wherein in course of step (3) the pH-value is adjusted to 6.5 to 8.5.

21. The process according to claim 17, wherein in course of step (5) the precipitate is dried by freeze drying.

22. The process according to claim 17, wherein the amine is a diamine or a triamine.

23. The process according to claim 17, wherein the acid is H₃PO₄.

24. The process according to claim 17, wherein the total amount of all components except water is from 5 to 40% by weight based on the total amount of all components of the aqueous mixture.

25. The process according to claim 17, wherein n is 2 or 3 and Mⁿ⁺ is selected from the group of Cu²⁺, Zn²⁺, Ni²⁺, Co²⁺, Ca²⁺, and Al³⁺.

26. The process according to claim 17, wherein n is 2 and Mⁿ⁺ is Cu²⁺.

27. Solid Mⁿ⁺(HDO)ₙ salts, wherein HDO is the anion of N′-hydroxy-N-cyclohexyl-diazenium oxide, Mⁿ⁺ is a metal cation with the exception of alkali metal cations and n has a value of 1-4 having a particle size D50 from 30 to 75 μm.

28. The solid salts according to claim 27 wherein the Mⁿ⁺(HDO)ₙ salts are selected from the group of Cu(HDO)₂, Zn(HDO)₂, Ni(HDO)₂, Co(HDO)₂, Ca(HDO)₂ and Al(HDO)₃.

29. The solid salts according to claim 27, wherein the salt is Cu(HDO)₂.

30. A method of protecting paints, coatings, construction materials, plastics and paper, leather, textiles, polymeric materials or surfaces comprising:
applying solid Mⁿ⁺(HDO)ₙ salts to the paints, coatings, construction materials, plastics and paper, leather, textiles, polymeric materials or surfaces.

31. The method of claim 30, wherein the Mⁿ⁺(HDO)ₙ salts are selected from the group consisting of: Cu(HDO)₂, Zn(HDO)₂, Ni(HDO)₂, Co(HDO)₂, Ca(HDO)₂ and Al(HDO)₃.

32. The method of claim 30, wherein the Mⁿ⁺(HDO)ₙ salt is Cu(HDO)₂.
